(19) United States
(12) Patent Application Publication
(10) Pub. No.: US 2009/0036400 A1
(43) Pub. Date: Feb. 5, 2009

(54) Pharmaceutical Preparations for Treating the Consequences of Alcohol Abuse, Hepatitis, Pancreatitis, Alzheimer's Disease, Parkinson's Disease, Diabetes, Toxic Kidney Disease, Reperfusion Damage, Arteriosclerosis, and as an Antidote Against Environmental Toxins and Medicinal Intoxication

(76) Inventors: Thomas Haehner, Dornstadt (DE); Dieter Mueller-Enoch, Dornstadt (DE)

Correspondence Address: BUCHANAN INGERSOLL & ROONEY PC, P.O. BOX 1404, ALEXANDRIA, VA, 22313-1404, US

(21) Appl. No.: 11/666,569

(22) PCT Filed: Oct. 28, 2005
(86) PCT No.: PCT/DE2005/001938

§ 371 Date: Dec. 17, 2007

(30) Foreign Application Priority Data
Oct. 29, 2004 (DE) 10 2004 052 697.4

(51) Int. Cl. A61K 31/662; C07F 9/28; A61K 31/715; C07H 1/00; A61P 25/32; A61P 25/28; A61P 25/00; A61P 13/12; A61P 3/10; A61P 9/10

(52) U.S. Cl. 514/54; 564/15; 514/114; 536/123.1

(57) ABSTRACT

A pharmaceutical preparation is disclosed for the prevention, treatment and relief from the consequences of alcohol abuse, viral hepatitis, steatohepatitis, acute and chronic pancreatitis, Alzheimer's disease, Parkinson's disease, toxic kidney diseases, acute kidney failure, toxic side effects on dosage of chemotherapeutics, diabetes mellitus, Wilson's disease, sideroses and/or ischaemic reperfusion damage, arteriosclerosis and as an antidote against environmental toxins and medicament intoxication. The preparation comprises at least one compound of general formula R-A-X, as active ingredient, where R=straight or branched chain C₅-C₂₇ alkyl with double and/or triple bonds and which can be substituted, A=—CO—, —COO—, —POₓ²⁻—, —NH_{y}—, —O— and/or —S—, x=2, 3 or 4, y=1 or 2 and X═H, polyol, amino acid, alkylamino, C₁- to C₃-alkylamino, C₁- to C₃ hydroxyalkyl and/or ₁- to C₃-carboxylic acid.

The invention relates to the use of lipophilic alkyl groups as well as hydrophilic radicals that have substances for the production of pharmaceutical preparations.

Excessive alcohol consumption on a sustained basis results in a liver disease, the so-called fatty liver, which can develop into an inflammation of the liver or hepatitis and, in the late stage, into a cirrhosis of the liver. In this case, the risk and the extent of the respective liver damage depends directly on the amount and the duration of the alcohol consumption, whereby the risk is different individually. An alcohol-induced inflammation of the liver (alcohol hepatitis) represents a potentially life-threatening disease, which can be accompanied by fever, jaundice, as well as an increase in white blood cells. Such alcohol-induced liver inflammations can be healed by complete abstinence from alcohol; cicatrizations in the case of cirrhoses of the liver are excluded. Since, however, alcohol abuse is frequently marked by a strong substance-abusing behavior, in practice a complete abstinence from alcohol is not easily achievable. In addition to this alcohol-induced thus-mentioned fatty-liver hepatitis or alcoholic steato-hepatitis (ASH), hepatitides also develop in individuals who do not abuse alcohol or do not partake of any alcohol at all. Such hepatitides are induced by, for example, environmental toxins, for example, in working in paint shops and/or also through medications.

Alzheimer's disease is a progressive dementia disease, which ultimately results in the complete loss of memory and personality. It is caused by protein deposits in nerve cells, plaques, which are formed from β-amyloid proteins or so-called τ-proteins. The actual cause has been unknown up until now, and both metabolic disorders, gene mutations, as well as so-called slow-virus infections or prions are discussed.

In the case of Alzheimer's disease, it is known that in the brains of transgenic mice, a trigger of the plaque formation is a lipid oxidation that leads to a G-amyloid precursor protein that forms the known plaque. Such a lipid oxidation is produced by a reactive oxygen species (ROS).

Parkinson's disease is a degeneration of dopaminergic neurons in the substantia nigra of the brain. In this case, this is the most common neurological disease in old age. Early signs are especially shaky movements here (tremors), a prevailing depressive mood, apathy as well as slowed thinking.

It is known that oxidation processes take place in metabolism with the aid of cytochromes. Cytochromes are a number of different enzymes whose active center has a hemostructure. In a number of oxidation reactions, they catalyze the transfer of electrons to an acceptor, which, for example, is molecular oxygen in cytochrome c, by which a radical superoxide anion (O₂⁻) is formed. The cytochrome c itself occurs in virtually all organisms that have a mitochondrial respiratory chain, i.e., in all plants and animals as well as in eukaryotic microorganisms. In this connection, it is advantageous that cytochrome c, during the evolution since its development, has hardly changed at all over 1.5 billion years. In addition to the cytochrome c, there are a number of other cytochromes, which differ very greatly from cytochrome c and perform the completely different oxidation or hydroxylation reactions in the metabolism. An important group in this connection is the cytochrome P450, which in turn is present in a number of completely different forms, such as 1A1, 2B1, 2C9, 2J2, 2E1, 3A1, etc.

Thus, for example, the cytochrome P 450 2B1 of the rat and human cytochrome P 450 2E1 are distinguished in their amino acid sequence in about 60%, i.e., both the structures of the active and catalytic centers as well as the size and shape of the access channels for the substrate are quite considerably different. This ultimately results in that both enzymes metabolize completely different substrate classes, i.e., the isoform 2E1 reacts with significantly smaller molecules, for example ethanol, acetone or else p-nitrophenol. This is quite characteristic of all isoforms of P 450 hemoproteins, i.e., one isoform therefore cannot be broken down by the other. These findings that are obtained with the isoform 2B1 of the rat thus cannot be transferred to the 2E1 isoform of the human.

It is known that both in alcohol consumption and in non-alcoholic fatty liver hepatitis, the synthesis of the cytochrome P450-2E1 is induced. The function and the mode of action of the latter of other cytochromes of greatly different isoforms is described by, for example, M. H. Wang et al. in Archives of Biochemistry and Biophysics, (1995) Vol.317, pages 299 to 304. Then, the enzyme has an approximately 15 Å long binding pocket, on whose end the reactive center sits with a hemoring that has a central iron atom.

In Z. Naturforsch. (2001) 56c, pages 1082-1090, Müiller-Enoch et al. describe the inhibition of cytochrome-P450 2B1 of the rat by means of lysophosphatidylcholines, lysophosphatidylinositol as well as arachidonic and oleic acids or by means of monoacyl glycerols, monooleyl glycerols, and monopalmitoyl glycerols.

In addition, T. Haehner, D. Müller-Enoch et al. in Z. Naturforschung (2004) 59c, pages 599-605 describe the influence of one-chain lipid molecules on the activity of the isoform cytochrome P 450 2B1 of the rat.

The invention has the purpose of eliminating the development and the consequences of alcoholic and non-alcoholic liver diseases, viral hepatitis, diabetes mellitus type 1 and type 2, pancreatitis, acute renal failure, toxic kidney diseases, side effects when using chemotherapeutic agents, Alzheimer's disease as well as Parkinson's disease, Wilson's disease, sideroses as well as arteriosclerosis and/or of attenuating its course and/or stopping it completely. The invention also has the purpose of alleviating and/or eliminating organ damage in ischemic conditions, e.g., in the case of reperfusion. Finally, the invention also has the purpose of treating the organotoxicity of environmental toxins and pharmaceutical substances.

This purpose is achieved by the features that are defined in the claims.

It was namely found, surprisingly enough, that in cytochrome P 450, in particular in the isoforms of group 2, especially 2E1, as well as 2C9, the formation of reactive oxygen species (ROS), in particular oxygen radicals as well as the superoxide anion and the hydroxyl radical (OH), which are not consumed in a direct redox reaction, can be prevented by administration of a substance that has a hydrophobic alkyl chain as well as a polar hydrophilic end. Such substances have a hydrophobic area and a hydrophilic area that are at least six carbon atoms long. These areas can be both synthetic one-chain lipids such as, for example, alkylphosphocholines, alkylcoumarins, or else alkylsulfonic acids, and natural one-chain lipids, such as corresponding fatty acids, fatty acid esters or sphingosine or lysophosphatidylinositol. This is all the more surprisingly known in that this isoform in its natural function is responsible for the oxidation of small molecules, such as ethanol and acetone.

The substances that are used according to the invention for the production of a pharmaceutical agent have in particular the general formula R-A-X. In this case, R represents an alkyl radical, which preferably has 7 to 27 C atoms. Especially preferred are alkyl radicals with a length of at least 8 C atoms, whereby a length of at least 10, in particular at least 11 or 12 C atoms is quite especially preferred. In addition, alkyl radicals with a maximum number of 20 C atoms are preferred, whereby a size of at most 18, in particular at most 17 or 16 C atoms, is quite especially preferred. The alkyl radical optionally can also be both straight-chain and branched and can contain one or more carbon double bonds and/or also triple bonds. The alkyl radical preferably has no more than 7 double bonds, however, in particular no more than 5 double bonds, and preferably contains no more then 3 triple bonds, in particular no more than 2 triple bonds. The alkyl radical can optionally be substituted with other groups, as long as the latter do not influence the hydrophobic behavior of the radical R. Preferred substituents are C₁-C₆-alkyl radicals, preferably C₁- to C₄-radicals, which themselves can also be branched, but preferably are straight-chain.

A represents a carbonyl, carboxylic acid, phosphoric acid, amino, ether or mercaptoether group, with the respective general formula —CO—, —COO—, POₓ²⁻, —NH_{Y}— and/or —S—.

X is a hydrogen atom, a polycyclic, aromatic or aliphatic, water-soluble hydrocarbon, which contains at least one heteroatom and which optionally is connected to the alkyl radical R under ring closure and which contains oxygen, nitrogen and/or sulfur heteroatoms to improve the water solubility, specifically in the ring and/or as—or in—the substituent. In addition, x stands for a polyol, an amino acid, a sugar, an alkylamine, a C₁- to C₃-alkylamine, a C₁- to C₃-hydroxyalkyl and/or a C₁- to C₃-carboxylic acid. As a polyol, all alkyl radicals that have multiple hydroxy groups are suitable, such as for example, glycol, glycerol and cyclic alcohols, such as, for example, inositol, sugar, as well as oligosaccharides and sugar radicals, which can be both one or more C₅-sugar or else C₆-sugar.

According to the invention, all substances that have detergent properties and that contain an alkyl or alkylene radical can be used. The substances can be both non-ionic, ionic and zwitterionic detergents and in particular have a critical micelle concentration (CMC) between 0.5*10⁻² M and 0.5*10⁻⁶ M.

Preferred substances according to the invention also comprise fatty acids, phosphoric ester, in particular phosphoric diester, fatty acid ester, in particular with alkylamines and amino acids, sphingosine and sphingosine derivatives, as well as cyclic alkyls and alkylene derivatives of general formula CH₃—R—X.

Fatty acids of general formula H₃C—R—COOH that can be used according to the invention can contain both unsaturated and saturated fatty acids, whereby R represents any hydrocarbon chain with up to 26 C atoms. Preferred chains R are —(CH₂)_{5 to 7} CH═CH(CH₂)₇—, —(CH₂)₇CH═CH(CH₂)₁₃—, —(CH₂)₃(CH₂CH═CH)_{2 to 4} (CH₂)_{7 to 3}—, —(CH₂CH═CH)_{3 to 6}(CH₂)_{7 to 2}—

Phosphodiesters that can be used according to the invention have the general formula H₃C—R—PO₄⁻—X, whereby R has an alkyl radical with 4 to 26 carbon atoms, which preferably is straight-chain. Especially preferred radicals R are —CH₂)_{4 to 24}—, —(CH₂)_{5 to 7}CH═CH(CH₂)₇—, —(CH₂)₃(CH₂CH═CH)_{2 to 4}(CH₂)_{7 to 3}—, —(CH₂CH═CH)_{3 to 6}(CH₂)_{7 to 2}—

whereby X is a hydrophilic radical or group that mediates water solubility and that has in particular heteroatoms such as N, S, or O and contains in particular amino, ammonium and hydroxy groups. X optionally can also represent hydrogen, so that a phosphomonoester develops.

Preferred radicals X are amino acids, in particular α, β, γ, and/or ω amino acids, as well as

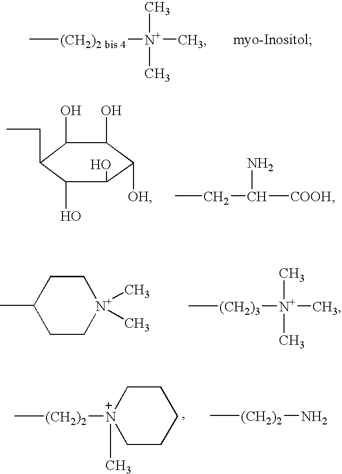

[Key: bis =to]

Preferred fatty acid amino acid esters are in particular carnitine esters of general formula

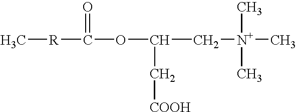

whereby R represents —CH₂)₆— to —(CH₂)₂₂—, —(CH₂)_{5 to 7}CH═CH(CH₂)₇—, —(CH₂)₃(CH₂CH═CH)_{2 to 4}(CH₂)_{7 to 3}—, —(CH₂CH═CH)_{3 to 6}(CH₂)_{7 to 2}—.

Other substances that can preferably be used according to the invention comprise sphingosines and sphingosine derivatives of general formula R-Pi-Y, whereby R represents a sphingosyl radical of general formula

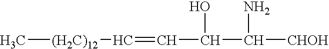

and Pi is a phosphate radical. Sphingosyl derivatives are defined as all structurally analogous alterations of the basic structure, such as, for example, a shortening or lengthening of the hydrocarbon chain, the addition of additional double bonds, hydroxy and/or amino groups or the exchange of these groups by H radicals. Preferred radical Y groups comprise hydrogen, choline, sugar radicals such as glucose, galactose as well as oligosaccharides and cyclic polyalcohols such as inositol, in particular myo-inositol.

In addition, in a preferred embodiment according to the invention, those substances can also be used whose hydrophilic group consists of a water-soluble, cyclic or polycyclic aromatic and/or aliphatic hydrocarbon radical, which contains in particular O, N and/or S to increase the solubility of the heteroatoms, especially in the ring and/or in a substituent thereof or as a substituent itself.

Also, cyclic alkyl and alkylene derivatives of general formula

H₃C—(R)—X,

whereby R is a C₄ to C₂₅ alkyl radical and in particular

—(CH₂)_{4 to 24}—, —(CH₂)_{5 to 7}CH═CH(CH₂)₇—, —(CH₂)₃(CH₂CH═CH)_{2 to 4}(CH₂)_{7 to 3}—, —(CH₂CH═CH)_{3 to 6}CH_{2 7 to 2}—,

and X is a water-soluble, cyclic hydrocarbon radical, which can be aliphatic or aromatic and the heteroatom has in particular O, N and/or S,
belong to the substances that can be used according to the invention. In this case, the cyclic hydrocarbons can be connected to the radical R under ring closure, as long as their hydrophobic properties in this case do not change. It is especially preferred in this case to suppress the formation of micellar structures by means of such cyclic hydrocarbons. Preferred compounds are phosphocholines, which in addition to their phosphocholine group have no esters, hydroxy or amino or amido groups or derivatives thereof. In an especially preferred embodiment, X is a phosphocholine radical and in particular a —PO₄⁻—R′—N⁺R₃″, whereby R′ can be a C₁-C₆-alkyl radical, in particular a C₁-C₃ radical, and R″, in each case independently, can be a hydrogen, methyl, ethyl and/or propyl radical.

Such a quite especially preferred substance is hexadecylphosphocholine. Finally, 1-O-octadecyl-2-O-methyl-sn-glycerol-3-phosphocholine is also still quite especially preferred. Such substances are described in, for example, Matzke et al. in the European Journal of Cell Biology 80, (2001), 1-10.

It has now been found, surprisingly enough, that with these substances, the consequences of alcohol abuse are eliminated and/or can be reduced. These are in particular liver damage or else other alcohol-induced, inflammatory processes. In addition to the pure alcohol-induced liver damage, diet-induced and endocrine factors, such as, e.g., adiposity, but also diabetes mellitus and hyperlipidemia, can also cause sever liver damage independently of alcohol, which can range from a fatty liver hepatitis (non-alcoholic steato-hepatitis=NASH) to cirrhosis of the liver. Such alcoholic and non-alcoholic fatty liver diseases often accompany a viral infection of the liver. In this case, it can result in a very quick progression of the disease. It has been shown that this can be attributed to, e.g., a synergistic production of reactive oxygen species (ROS) and the associated cell damage. All previously mentioned diseases or their causes or consequences can be treated with the inventive means.

Moreover, alcohol abuse also results in the damage of additional organs, such as, for example, the pancreas, the heart or the nervous system.

It has also been found that these substances are also especially well suited for treating inflammations of the pancreas. In addition to alcohol abuse, such inflammations or pancreatitides can also be caused by toxic substances. These include in particular environmental toxins, such as professional chemicals, or else medications. Also, viral infections or metabolic-endocrine factors can cause such pancreatic inflammations, whereby in all cases, reactive oxygen species are involved in the development of disease and in the progression of the disease.

Even in the treatment of diabetes mellitus, both in diabetes mellitus type 2 and type 1, the pharmaceutical agent according to the invention has proven suitable. It has namely been shown that β-islet cells are especially sensitive compared to oxidative processes and that the latter quickly decrease in the case of increased oxidative stress. This oxidative stress can be avoided with the pharmaceutical agent according to the invention, but is at least massively reduced.

Also, in the treatment of Alzheimer's disease, as well as in Parkinson's disease, the pharmaceutical agent according to the invention has proven effective. In this case, it has been shown, for example, that with the substances according to the invention, the concentration of dopamine can be increased by reduced degradation.

Also, toxic nephropathies, as well as other diseases, as they are caused by, e.g., side effects in the administration of chemotherapeutic agents, in particular cell toxins, such as metal complexes like cisplatin, carboplatin, titanocene dichloride or gold complexes, can be treated with the pharmaceutical agents according to the invention. In this connection, it has been shown in particular that the organotoxicity of metal complexes or else other toxic agents, such as halogenated hydrocarbons and namely both mono-halogenated and poly-halogenated hydrocarbons below also can prevent vapor narcotics of the halothane type, as well as corresponding aromatic hydrocarbons, nitrosamine, acrylamide or pharmaceutical agents, such as paracetamol, methotrexate, isoniazide or aminoglycoride-antibotics or x-ray contrast media. The pharmaceutical agent according to the invention is thus also suitable for treatment of organotoxicity of environmental toxins, in particular as an antidote for this purpose in organs such as the liver, kidneys, central nervous system, pancreas, etc.

Also suitable is the pharmaceutical preparation according to the invention for treating acute renal failure, in particular that renal failure that was caused by medicinal intoxication, hemolytic diseases, the hemolytic-uremic syndrome (Gasser Syndrome), rhabdomyolysis (breakdown of the fasciated skeletal muscles), by circulatory-ischemic processes and/or a viral interaction. Moreover, it has proven its value for the treatment of damage that has proven its value by crushing the fasciated muscles (crush syndrome) and/or their breakdown in the administration of medications (such as, e.g., CSE inhibitors, e.g., Lipobay).

It has proven quite especially suitable to avoid damage that results by reperfusion of biological tissues, such as, for example after organ infarctions, in particular the heart, as well as the brain (myocardial infarction, stroke). Thus, for example, it could be shown in animal experiments that such reperfusion damages contribute to 60-80% of the tissue destruction or that the propagation of the tissue death can be reduced by this factor. The agent according to the invention is thus also especially suitable for avoiding reperfusion damage in transplanted organs. Such organs are kept in a cooled nutrient solution until their transplantation in the body of a new recipient. After the transplantation and after connection to the circulatory system of the recipient, bodily fluids then flow through the recipient again, which results in reperfusion damage. By an administration of the agent according to the invention before and during the positioning as well as shortly before the implantation in the recipient organism, this important transplantation problem can also be resolved.

The substances according to the invention have proven of value in particular as inhibitors of the human isoforms of the gene family 2 of the cytochrome P450, namely in particular of the isoforms 2E1 and 2C9 and by these induced diseases.

### EXAMPLE 1

### Enzyme Preparations:

8 male Sprague-Dawley rats were administered 5% acetone (v-v) in drinking water for 7 days, as described by M. H. Wang et al. in Arch. Biochem. Biophys. 317: 299-304, 1995 to achieve a cytochrome P450 2E1 induction. The preparation of the microsomal fraction is carried out based on the method published by Wang, P., Mason, P. S., Günderlich, F. G., in Arch. Biochem. Biophys. 1999: 206, 1980. In comparison to control rats, in this case, an increase of the p-nitrophenol-hydroxylase activity by the factor 5 could be determined.

### Enzyme Activity Determinations:

p-Nitrophenol applies as a specific substrate for cytochrome P450 2E1. The determination of the p-nitrophenol-hydroxylase activity was performed based on the work by J. W. Allison and B. L. Robinson, Anal. Biochem. 219,49-52, 1994.

In the case, the following inhibiting constants were determined:

| | |
|---|---|
| Dodecylphosphorylcholine | Kᵢ = 0.45 μm |
| Hexadecylphosphocholine (miltefosin) | Kᵢ = 0.60 μm |
| rac-1-O-phosphocholine-2-amino-octadecane-hydrochloride | Kᵢ = 2.25 μm |

In all cases, a competitive inhibition can be noted.

### Detection of Oxygen Radicals:

To this end, a microsomal fraction from a human liver was obtained as is described by M. H. Wang et al. in the previously mentioned work. The cytochrome P450-mediated oxygen production was determined by fluorimetry with the aid of the dichlorofluorescein method (BASF, B. A. et al., Journal of Immunology 130 (4), p. 1910, 1983). The detection is carried out based on different specific substrates of the isoform of the gene family 2, e.g., ChIP 2C9: Diclofenac, ChIP 2C1: p-nitrophenol) and based on various inhibitory supporting substances (e.g., alkylphosphocholines, sphingolipids, lysophospholipids). In all cases, a concentration-dependent reduction of the ROS-mediated dichlorofluorescein fluorescence occurred (complete suppression in the range of about 5-15 μm).

1. Use of the compounds of general formula R-A-X for the production of a pharmaceutical preparation for prevention, treatment and relief from the consequences of alcohol abuse, viral hepatitis, steato-hepatitis, acute and chronic pancreatitis, Alzheimer's disease, Parkinson's disease, toxic kidney diseases, acute renal failure, toxic side effects in the administration of chemotherapeutic agents, diabetes mellitus, Wilson's disease, sideroses and/or ischemic reperfusion damage, arteriosclerosis and as an antidote against environmental toxins and medicinal intoxication, characterized in that in the general formula
R-A-X
R represents a C₅-C₂₇-alkyl radical, which is straight-chain or branched, which can have double and/or triple bonds, and which can be substituted, and
A— is a —CO—, —COO—, —POₓ²⁻—, —NH_{y}—, —O— and/or —S—, and x means 2, 3 or 4, and y means 1 or 2, and
X is a hydrogen atom, a polyol, an amino acid, an alkylamine, a C₁- to C₃-alkylamine, a C₁- to C₃-hydroxyalkyl and/or a C₁- to C₃-carboxylic acid.

2. Use according to one of the preceding claims, wherein X is a choline, myoinositolyl, glucosyl, galactosyl radical or an oligosaccharide.

3. Use according to one of the preceding claims, wherein X is an α, β, γ, and/or Ω amino acid.

4. Use according to one of the preceding claims, wherein X is an α, β, or γ-hydroxyamino acid.

5. Use according to one of the preceding claims, wherein the inhibitor is a nonionic, ionic or zwitterionic detergent with a critical micelle concentration of 0.5*10⁻² M to 0.5*10⁻⁶ M.

6. Pharmaceutical preparation that contains at least one substance in which in the general formula
R-A-X
R represents a C₅-C₂₇-alkyl radical, which is straight-chain or branched, which can have double and/or triple bonds, and which can be substituted, and
A— is a —CO—, —COO—, POₓ²⁻—, —NH_{y}—, —O— and/or —S—, and x means 2, 3 or 4, and y means 1 or 2, and
X is a hydrogen atom, a polyol, an amino acid, an alkylamine, a C₁- to C₃-alkylamine, a C₁- to C₃-hydroxyalkyl and/or a C₁- to C₃-carboxylic acid optionally together with other pharmaceutical excipients.
